(19) United States
(12) Patent Application Publication
(10) Pub. No.: US 2026/0335100 A1
(43) Pub. Date: Oct. 29, 2026

(54) IMPLANT FOR STABILIZING ADJACENT VERTEBRAE

(71) Applicant: Advance Research System, LLC, Edina, MN (US)

(72) Inventor: Hamid R. Abbasi, Minneapolis, MN (US)

(21) Appl. No.: 19/657,936

(22) Filed: Apr. 24, 2026

Related U.S. Application Data
(60) Provisional application No. 63/794,121, filed on Apr. 24, 2025.

(51) Int. Cl. A61F 2/44; A61F 2/46

(52) U.S. Cl. CPC A61F 2/4425; A61F 2/4611; A61F 2002/443; A61F 2210/0061

(57) ABSTRACT

An implant for placement between adjacent spinous processes includes a first adjustable member and a second adjustable member, each comprising a superior arm and an inferior arm having surfaces configured to contact at least a portion of adjacent vertebrae. The superior arm and inferior arm define an adjustable spacing therebetween. One or more coupling members extend between and couple the first and second adjustable members, configured to permit bone growth therethrough. An adjustment mechanism is operably coupled to at least one adjustable member and configured to increase or decrease the spacing at an anterior end. The implant allows for controlled distraction and stabilization of adjacent vertebrae while promoting fusion.

### CROSS REFERENCES TO RELATED APPLICATION

This application claims the benefit of U.S. Provisional Application No. 63/794,121, filed Apr. 24, 2025, which is herein incorporated by reference in its entirety for all purposes.

### TECHNICAL FIELD

The present disclosure relates to spinal implant devices, and more particularly to an adjustable interspinous process device for stabilizing adjacent vertebrae.

### BACKGROUND

Spinal disorders and degenerative conditions affecting the spine are common causes of pain and disability. As people age, the structures of the spine, including vertebrae, intervertebral discs, and ligaments, may undergo changes that can lead to instability, nerve compression, and reduced mobility. These issues often result in chronic back pain, reduced quality of life, and limitations in daily activities for many individuals.

Traditional treatments for spinal disorders range from conservative approaches like physical therapy and medication to invasive surgical procedures such as spinal fusion. However, there is a growing interest in minimally invasive techniques that can provide stabilization and pain relief while preserving some degree of natural spinal motion. Interspinous process devices have emerged as one such option, offering potential benefits in terms of reduced surgical trauma and faster recovery times compared to more extensive spinal surgeries. These devices aim to address the challenges of maintaining proper spacing between vertebrae, redistributing loads, and potentially promoting bone growth in affected spinal segments.

### SUMMARY

In some aspects, the techniques described herein relate to an implant for placement between adjacent vertebrae, including: a first adjustable member and a second adjustable member, each of the first adjustable member and the second adjustable member including: a superior arm and an inferior arm, each of the superior arm and the inferior arm having one or more surfaces configured to contact one or more vertebrae; an anterior end configured for insertion between the adjacent vertebrae; and a posterior end opposite the anterior end, wherein the superior arm and the inferior arm define an anterior spacing at the anterior end and a posterior spacing at the posterior end, and wherein the anterior spacing is adjustable relative to the posterior spacing; one or more coupling members extending between and coupled to the first adjustable member and the second adjustable member, the one or more coupling members configured to permit bone growth therethrough; and an adjustment mechanism operably coupled to at least one of the first adjustable member and the second adjustable member and configured to increase or decrease the anterior spacing.

In some aspects, the techniques described herein relate to a method for implanting a spinal implant between adjacent vertebrae, the method including: inserting an implant between adjacent spinous processes of the adjacent vertebrae, the implant including: a first adjustable member and a second adjustable member, each of the first adjustable member and the second adjustable member including a superior arm and an inferior arm, an anterior end, and a posterior end; one or more coupling members extending between and coupled to the first adjustable member and the second adjustable member; and an adjustment mechanism operably coupled to at least one of the first adjustable member and the second adjustable member; actuating the adjustment mechanism to increase or decrease an anterior spacing between the superior arm and the inferior arm at the anterior end; and positioning at least one of the inferior arm of the first adjustable member or the inferior arm of the second adjustable member in contact with a lamina of a vertebra.

In some aspects, the techniques described herein relate to a system for stabilizing adjacent vertebrae, including: an implant configured for placement between adjacent spinous processes of the adjacent vertebrae, the implant including: a first adjustable member and a second adjustable member, each of the first adjustable member and the second adjustable member including a superior arm and an inferior arm, an anterior end, and a posterior end; one or more coupling members extending between and coupled to the first adjustable member and the second adjustable member, the one or more coupling members configured to permit bone growth therethrough; and an adjustment mechanism operably coupled to at least one of the first adjustable member and the second adjustable member and configured to adjust an anterior spacing at the anterior end; and an instrument configured to engage the implant and actuate the adjustment mechanism during implantation.

### BRIEF DESCRIPTION OF THE DRAWINGS

The disclosure can be understood in consideration of the following detailed description of various embodiments in connection with the accompanying drawings. Non-limiting and non-exhaustive examples are described with reference to the following figures.

FIG. 1 is a conceptual diagram illustrating side sectional views of an example spinal implant as a sequence of steps for positioning and deploying the spinal implant between adjacent vertebrae.

FIG. 2 is a conceptual diagram illustrating a perspective view of an example spinal implant system with adjustable members and an insertion instrument.

FIG. 3 is a conceptual diagram illustrating an exploded view of the components of the spinal implant system of FIG. 2.

FIG. 4 is a conceptual diagram illustrating a perspective view of an example spinal implant device with adjustable members and a coupling member.

FIG. 5 is a conceptual diagram illustrating an exploded perspective view of an example adjustable member showing internal components and surfaces.

FIG. 6 is a conceptual diagram illustrating a side view of an example spinal implant device having the internal configuration of an adjustable member with a first vertical spacing between the superior arm and the inferior arm at the anterior end.

FIG. 7 is a conceptual diagram illustrating a side view of an example spinal implant device having the internal configuration of an adjustable member with a second vertical spacing between the superior arm and the inferior arm at the anterior end, the second vertical spacing being greater than the first vertical spacing of FIG. 6.

FIG. 8 is a conceptual diagram illustrating a reverse side view of the adjustable member of the spinal implant device of FIG. 7.

FIG. 9 is a conceptual diagram illustrating a side view of an example spinal implant device with adjustable members and a coupling member.

FIG. 10 is a conceptual diagram illustrating a sectional view B-B of the spinal implant device of FIG. 9.

FIG. 11 is a conceptual diagram illustrating an anterior end view of the spinal implant device of FIG. 9.

FIG. 12 is a conceptual diagram illustrating a top view of the spinal implant device of FIG. 9.

FIG. 13 is a conceptual diagram illustrating a sectional view A-A of the spinal implant device of FIG. 12 showing the internal configuration of an adjustable member.

FIG. 14 is a conceptual diagram illustrating a detailed view of a portion of the spinal implant device of FIG. 12 of a coupling member with a mesh structure.

### DETAILED DESCRIPTION

For purposes of promoting an understanding of the principles of the disclosure, reference will now be made to the embodiments illustrated in the drawings, and specific language will be used to describe the same. It will nonetheless be understood that no limitation of the scope of the disclosure is intended by the illustration and description of certain embodiments of the disclosure. In addition, any alterations and/or modifications of the illustrated and/or described embodiment(s) are contemplated as being within the scope of the present disclosure. Further, any other applications of the principles of the disclosure, as illustrated and/or described herein, as would normally occur to one skilled in the art to which the disclosure pertains, are contemplated as being within the scope of the present disclosure.

The present disclosure relates to spinal implant devices for stabilizing adjacent vertebrae, and more particularly to implantable devices configured for insertion between spinous processes. The spinal implant devices described herein span between adjacent spinous processes and may provide support, promote bone growth, enable controlled distraction of vertebrae, or combinations thereof.

In some embodiments, the devices address limitations of existing interspinous process devices by distributing forces more evenly across spinous processes, potentially reducing the risk of bone erosion or fracture associated with point loading.

The devices may include one or more adjustable members, one or more coupling members, and one or more adjustment mechanisms. The coupling members may permit bone growth therethrough while maintaining structural support. The adjustable members may engage with anatomical structures and be actuated in situ using the adjustment mechanism to customize the implant geometry to patient anatomy. In some implementations, the implant may be inserted in a compact configuration and, once positioned in the anatomy of the patient, deployed to an expanded configuration. The implant devices described herein may be used to treat various spinal conditions including spinal stenosis. By providing stabilization and promoting fusion between adjacent vertebrae, the devices may help alleviate nerve compression and associated symptoms. The devices may offer minimally invasive treatment options compared to more extensive surgical procedures.

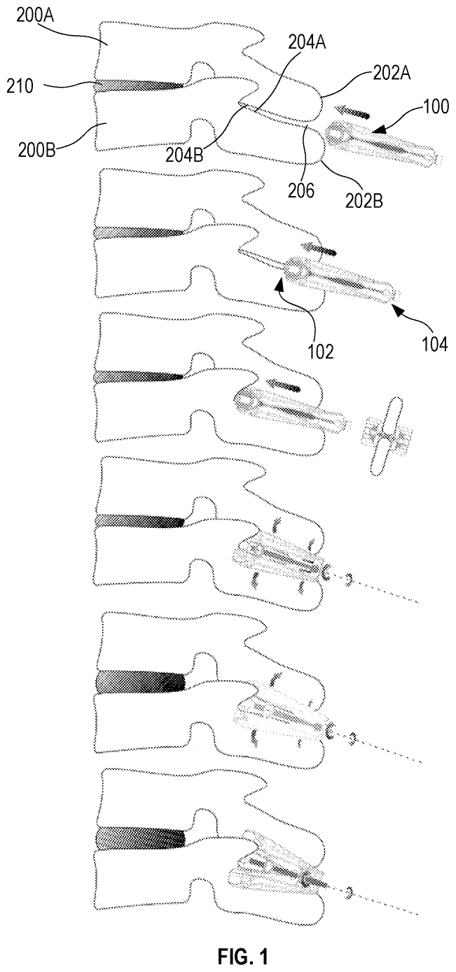

FIG. 1 is a conceptual diagram illustrating side sectional views of an example spinal implant as a sequence of steps for positioning and deploying the spinal implant between adjacent vertebrae. The anatomical relationship between a first vertebra 200A and a second vertebra 200B with an intervertebral disc 210 positioned between the vertebral bodies is shown. A first lamina 204A of first vertebra 200A and a second lamina 204B of second vertebra 200B are connected to a first spinal process 202A and a second spinal process 202B, respectively.

The spinal implant 100 (hereinafter, implant 100) may be positioned between first spinous process 202A of first vertebra 200A and second spinous process 202B of second vertebra 200B within an interspinous space 206. An anterior end 102 of implant 100 may serve as the insertion end of implant 100, and a posterior end 104 of implant 100 is positioned opposite of anterior end 102. Implant 100 may have a wedge-shaped profile that facilitates insertion and distraction of the vertebrae.

Implant 100 is shown in various stages of deployment (e.g., increasing vertical spacing between the superior arm(s) and inferior arm(s)), demonstrating a progressive adjustment sequence from top to bottom in the figure. This progressive adjustment sequence illustrates the range of motion and positioning capabilities of implant 100 when installed between first vertebra 200A and second vertebra 200B. In the uppermost depiction, implant 100 is shown before insertion between first vertebra 200A and second vertebra 200B. Below that, implant 100 is inserted between first spinous processes 202A and second spinous process 202B. In the next depiction, implant 100 is inserted further and may approach or contact an anatomical structure (e.g., a facet joint or a lamina, such as first lamina 204A and/or second lamina 204B, depending on the depth and angle of insertion). Subsequent views show implant 100 in progressively more open configurations, activated by the rotation of an actuation instrument. Each successive depiction illustrates increased anterior vertical opening of implant 100 to define a wedge shape, highlighting its adjustability.

In some cases, coupling member(s) may be configured to preferentially expand implant 100 in a direction biased toward the lamina and foramen, rather than uniformly or posteriorly toward the soft tissue adjacent the spinous process. The asymmetric or anatomically directed expansion may serve to open the neural foramen more effectively and promote a more lordotic spinal alignment. By directing the distraction force anteriorly and inferiorly, the implant may reduce impingement on posterior structures while achieving decompression and sagittal plane correction.

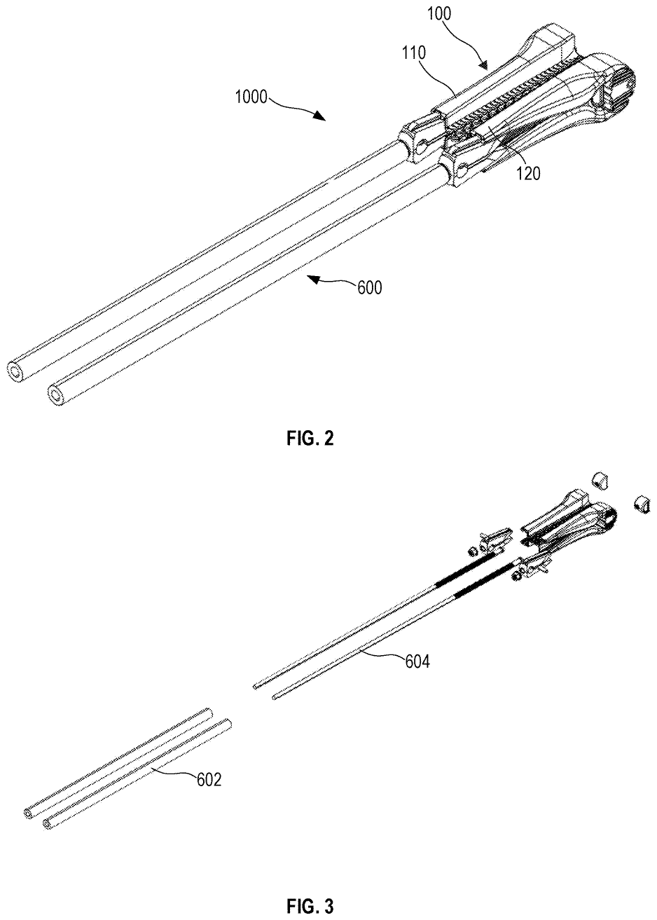

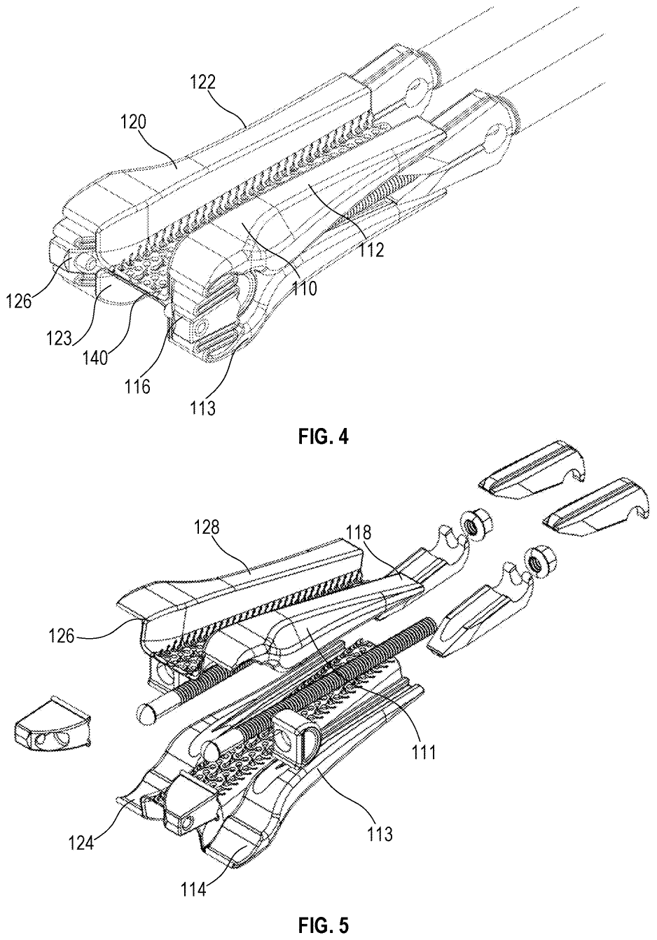

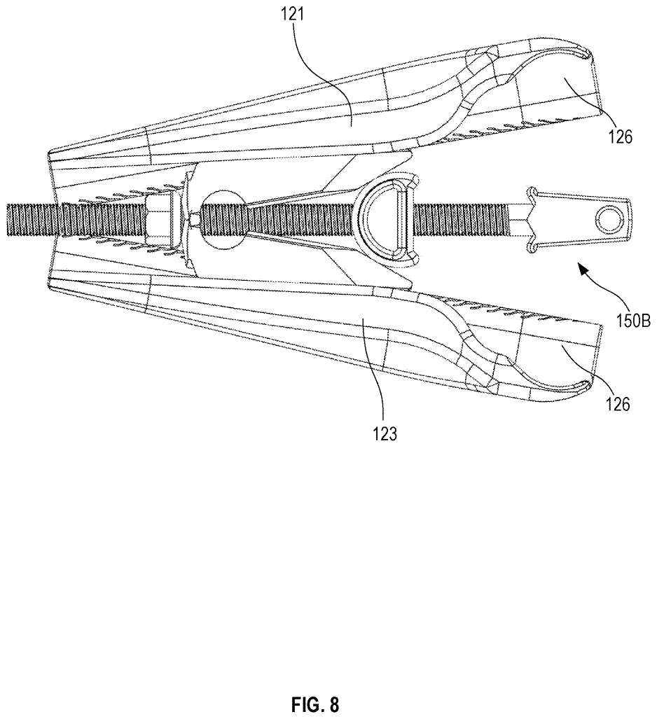

As illustrated in FIGS. 2 through 14, a spinal implant system 1000 may include one or more implants 100 and one or more instruments for operating implant(s) 100. Implant 100 may include one or more adjustable members, one or more coupling members, and one or more adjustment mechanisms. The adjustable members may be configured to receive and distribute compressive forces from adjacent spinous processes when implanted, potentially reducing the risk of bone erosion or fraction.

Implant 100 may include adjustable members, such as a first adjustable member 110 and a second adjustable member 120, as shown in FIGS. 2 through 5. In some cases, the anterior end of first adjustable member 110 and second adjustable member 120 may be configured for insertion between adjacent vertebrae. The superior surfaces 118, 128 of the first adjustable member 110 and the second adjustable member 120 may contact the superior vertebra (e.g., 200A). In some cases, first adjustable member 110 and second adjustable member 120 may each include a superior arm 112, 122 and an inferior arm 113, 123. The adjustable members may have a wedge-shaped profile with greater spacing at the anterior end compared to the posterior end. The configuration may allow for controlled distraction of the vertebrae. The superior 112, 122 and inferior arms 113, 123 of each adjustable member may define a spacing along a longitudinal axis between an anterior end and a posterior end. The superior 112, 122 and inferior arms 113, 123 of each adjustable member 110, 120 may define a vertical spacing at each of the anterior end and the posterior end. The vertical spacing between the superior arm 112, 122 and the inferior arm 113, 123 of each adjustable member may have a maximum at the anterior end and a minimum at the posterior end.

In some cases, the superior arm 112, 122 and the inferior arm 113, 123 may define a cavity 116, 126, which can house the adjustment mechanism 150A, 150B, bone graft, bone, etc. In some cases, the superior arm 112, 122 and the inferior arm 113, 123 may have textured surfaces configured to engage with the spinous processes or laminae of adjacent vertebrae. These textured surfaces may enhance fixation and stability of implant 100 when implanted. In some examples, at least one of the inferior arms may be contoured to engage a lamina of the inferior vertebra, thereby expanding the load-bearing interface and reducing the risk of spinous process fracture.

Implant 100 may include an adjustment mechanism 150A operably coupled to at least one of first adjustable member 110 or second adjustable member 120 as shown in FIGS. 5 through 8. Adjustment mechanism 150A may be configured to increase or decrease the spacing at the anterior end. The adjustability may allow for customization of the implant to fit various patient anatomies and provide the desired level of distraction.

In some cases, one or more coupling members 140 may extend between and couple first adjustable member 110 and second adjustable member 120, as illustrated in FIGS. 2 through 5, 10, 12, and 14. Coupling member(s) 140 may be configured to distribute compressive forces from and/or between first adjustable member 110 and second adjustable member 120. Additionally, coupling member(s) 140 may be designed to permit bone growth therethrough, potentially promoting fusion between adjacent vertebrae.

The combination of adjustable members, coupling members, and the adjustment mechanism may allow implant 100 to provide customizable distraction and stabilization while promoting bone growth and fusion. The ability to adjust the spacing at the anterior end relative to the posterior end may enable fine-tuning of the implant's fit and effectiveness in situ.

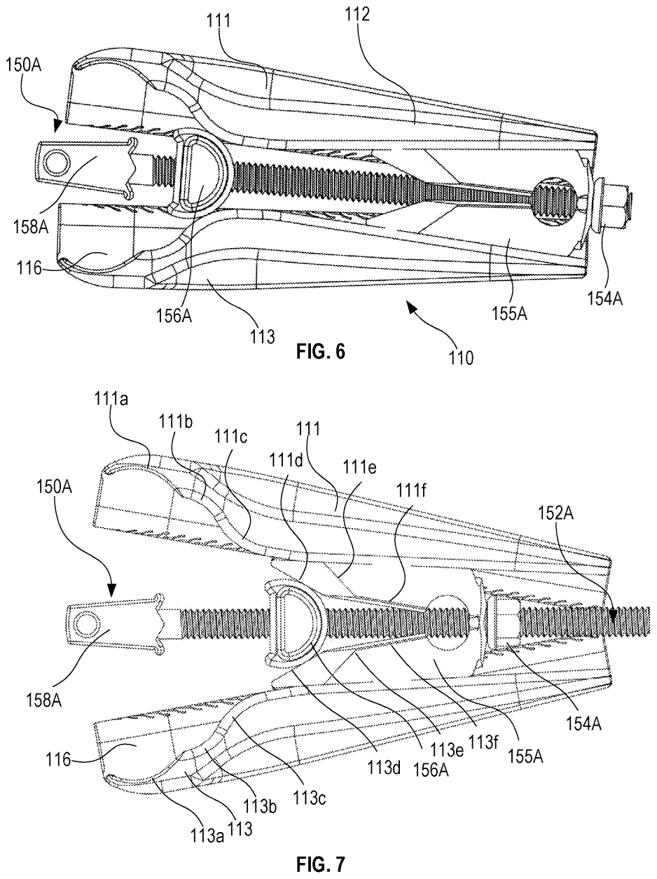

Implant 100 may include an adjustment mechanism 150A operably coupled to at least one of first adjustable member 110 or second adjustable member 120. As shown in FIGS. 6 through 8, adjustment mechanism 150A may include a threaded shaft 152A, an adjustment nut 154, a first wedge member 155A, a second wedge member 156A, and an end cap 158A. In some cases, threaded shaft 152A may extend through first adjustable member 110. Adjustment nut 154A may be engaged with threaded shaft 152A and configured to translate along threaded shaft 152A when rotated by an instrument, such as an insertion instrument 600. First wedge member 155A and second wedge member 156A may be positioned along threaded shaft 152A and configured to translate relative to one another. First wedge member 155A and second wedge member 156A may be configured to move along opposing inclined surfaces defined by first adjustable member 110 or second adjustable member 120. This movement may adjust the spacing between the superior arm and the inferior arm of the respective adjustable member. In some cases, rotation of threaded shaft 152A may cause first wedge member 155A and second wedge member 156A to move in opposite directions along a longitudinal axis of implant 100.

Adjustment mechanism 150A may interact with the curved or inclined lateral edges of the superior and inferior arms during expansion of the implant. For example, wedge members may sequentially engage the different regions of the multi-slope inner contour. Adjustment mechanism 150A may be configured to adjust a vertical spacing at the anterior end of first adjustable member 110. As wedge members translate, superior arm 112 and inferior arm 113 may be pushed apart, increasing the spacing between them.

End cap 158A may be positioned at one end of the adjustment mechanism 150A. End cap 158A may secure the components within the respective adjustable member 110. The end cap 158A may also serve to protect the internal components of the adjustment mechanism 150 from external debris or tissue ingrowth. In some examples, each of the end caps 158A and 158B may be coupled to one another, e.g., via a cross bar or the like, to provide lateral stability for each of the threaded shafts of the respective adjustable members. In some examples, end cap 158A may serve to limit the range of movement of the second wedge member 156A, thereby defining the maximum expansion of the adjustable member.

Second adjustment member 120 may have an adjustment mechanism that is similar to first adjustment mechanism 150A. Each of first adjustable member 110 and second adjustable member 120 may be adjusted either synchronously (e.g., vertical spacing between the superior and inferior arms is the same at corresponding positions along each member) or asynchronously (e.g., vertical spacing between the arms differs at corresponding positions along each member).

Alternatively, or additionally, the adjustment mechanism 150A may include a screw head having a drive interface, such as, for example, a torx drive, a lobed drive, a hex drive, or the like. The drive interface may be positioned at the posterior end of threaded shaft 152A. The drive geometry may be configured to interface with a complementary bit, providing better grip and control during adjustment. The screw head may act as a torque-limiting feature, preventing over-tightening of the mechanism, allow for quick engagement/disengagement of the adjustment instrument, or both.

Adjustment mechanism 150A may also include a ratcheting element or a cam in addition to or instead of the threaded screw configuration. A ratcheting mechanism could be incorporated to allow for incremental, unidirectional adjustment of the implant height. A cam could be used to convert rotational motion into linear motion for adjusting the implant height.

A ratcheting element may include a series of teeth or notches along a shaft or rod, etc. (e.g., threaded shaft 152A, wedge members 155A, 156A, etc.), interacting with a pawl or catch mechanism. This configuration may allow for incremental adjustment of the implant height in small, precise steps. The ratchet typically allows movement in one direction while preventing reverse motion, thereby maintaining the adjusted position. Each “click” of the ratchet may provide audible and tactile feedback to the surgeon, confirming adjustment. Ratcheting may also allow for faster initial positioning compared to threaded mechanisms.

A cam-based adjustment mechanism may utilize an eccentric or spiral-shaped component (which can be attached to the end of the threaded shaft) that rotates to drive the expansion of the implant. The cam profile can be designed to provide variable rates of expansion throughout the adjustment range, offering smooth and continuous adjustment without discrete steps. Cam mechanisms can be optimized to provide increased force multiplication at certain points in the expansion process and may allow for efficient use of space within the implant.

In some implementations, these alternative adjustment mechanisms may be integrated with the wedge members, allowing them to advance in discrete increments as the ratchet is actuated or as the cam is rotated. The ratcheting element or cam may be configured to directly push apart the superior and inferior arms, or they may drive the movement of wedge members to achieve expansion.

These alternative adjustment mechanisms may also be combined with the threaded screw configuration to create hybrid systems that provide both coarse and fine adjustment capabilities. For example, the implant may incorporate a switching mechanism to alternate between different adjustment modes, such as using a ratchet for quick deployment followed by a threaded component for final positioning.

In some embodiments, each of the superior and inferior arms (e.g., 111, 113) of each adjustable expansion member may define a multi-slope inner contour or surfaces extending along a longitudinal axis from an anterior end to a posterior end of the implant. These contours may include a series of distinct regions or surfaces configured to sequentially engage one or more wedge members (e.g., wedge members 155A and 156A) during actuation of an adjustment mechanism (e.g., threaded shaft 154A), thereby producing controlled vertical expansion between the arms. The inner contour may include a sequence of surfaces or regions having different geometries, such as linear, inclined, curved, or stepped profiles, arranged along a longitudinal axis extending from the anterior end toward the posterior end.

The contour may include one or more regions, such as an entry taper, a primary ramp, a secondary ramp, a mid-span plateau, a curved transition, and a posterior stabilization ramp. These surfaces may be configured to sequentially engage components of the adjustment mechanism (e.g.,150A) during actuation.

The multi-slope inner contour may include, for example:
(1) Entry taper: An anterior-most region defining a shallow or beveled surface. This region may serve as a lead-in or alignment guide for second wedge member 156A, facilitating initial engagement and reducing insertion resistance. The entry taper may be configured to center the wedge relative to the arms and ensure smooth advancement into the implant.
(2) Primary ramp: A steeper inclined surface (as compared to the entry taper) located immediately posterior to the entry taper. The primary ramp may be configured to convert anterior translational movement of second wedge member 156A into vertical separation between the superior and inferior arms. This region may provide a high mechanical advantage and may be primarily responsible for initiating distraction.
(3) Secondary ramp: A surface of moderate incline following the primary ramp. The secondary ramp may continue the separation of the arms at a reduced slope (at a more gradual rate), helping to regulate expansion force and maintain control over the spacing geometry. The secondary ramp can reduce peak mechanical stress and allow for fine-tuned control of implant height.
(4) Mid-span plateau: A substantially constant or low-incline region that may extend across the mid-body of the contour. In some examples, a region of relatively constant vertical spacing or low incline may be included between the ramps. This plateau may serve as a dwell zone, providing stable support as the wedge transitions from the initial expansion region to the posterior zone. In some examples, the plateau may assist in distributing compressive loads or stabilizing partial expansion during incremental actuation.
(5) Curved transition: A smooth concave surface that transitions between inclined regions or toward a flatter posterior profile. The curved transition may reduce frictional resistance, mitigate mechanical binding, and help guide the wedge during the latter phase of movement. This region may be configured to reduce binding or resistance during the latter stages of wedge translation. The curve may also promote self-centering and accommodate conformal engagement between the wedge and the arms.
(6) Posterior stabilization zone: The posterior stabilization zone may be a terminal region at or near the posterior end of the arm, which may have a reduced incline (e.g., generally flat) or even a reverse taper. This region may be configured to receive and stabilize the wedge member 156A in its fully actuated position (e.g., final expanded configuration, maximum vertical spacing). The stabilization zone may resist unintentional back-driving of the wedge member 156A and may serve as a locking surface to maintain the implant's final expanded geometry.

Inferior arm 113 may include complementary contour regions (e.g., 113a-113f), each corresponding in shape or function to those of the superior arm. In some embodiments, the superior and inferior contours may be symmetric, while in other embodiments, they may have differing geometries to accommodate anatomical structures or desired force distribution profiles.

The contour regions may be mirrored or complementary on the superior and inferior arms, and the geometry of each region may be selected based on the desired distraction profile, mechanical resistance, and engagement sequence with the wedge members. In some embodiments, the inner contour surfaces may be shaped to match or track the outer contour of the wedge members to facilitate consistent spacing and reduce mechanical wear.

During use, wedge member 156A may be inserted between the arms from an anterior access path and advanced posteriorly along a guide or actuator (e.g., threaded shaft 154A). Wedge member 156A may be partially received within wedge member 155A, such that the two wedge members translate relative to each other along opposing inclined surfaces. As wedge member 156A moves from anterior end to the posterior end, its upper and lower surfaces may sequentially engage contour regions 111a-111f and 113a-113f, producing progressive distraction of the arms.

This multi-stage interaction may allow the implant to expand in a controlled, stepwise manner, with varying rates of lift, force distribution, and positional stability provided by the different contour regions. The nested or interlocking configuration of wedge members 155A and 156A may further enhance mechanical stability and enable fine positional control during and after implantation.

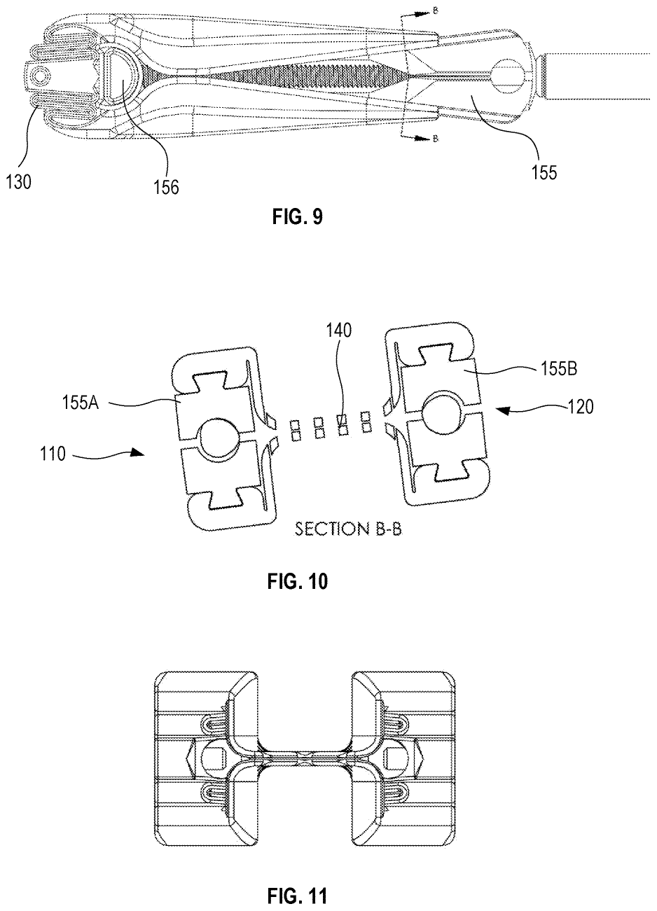

In some implementations, the adjustable members may incorporate surface stops to limit or control the extent of expansion. To prevent potential slippage of the wedge members, a dovetail feature may be incorporated into the design of the first wedge member (e.g., 155A) of the first adjustable member (e.g., 110) and/or first wedge member (e.g., 155B) of the second adjustable member (e.g., 120) (see FIGS. 9 and 10), the second wedge member (e.g., 156A), or the internal surfaces of the adjustable members. The dovetail feature may provide additional stability and prevent unintended movement of the components during adjustment or under a load.

Implant 100 may include a coupling member 140 extending between and coupled to first adjustable member 110 and second adjustable member 120. The coupling may distribute these forces across a larger surface area, similar to the way a hammock distributes weight. In some cases, coupling member(s) 140 may include a flexible mesh structure, such as a titanium mesh. The titanium mesh may provide strength and durability while maintaining flexibility. In other cases, coupling member 140 may be constructed from PEEK (polyetheretherketone) or a resorbable polymer. In some cases, coupling member(s) 140 may be made of fabric. The choice of material may depend on factors such as desired mechanical properties, biocompatibility, and long-term performance requirements.

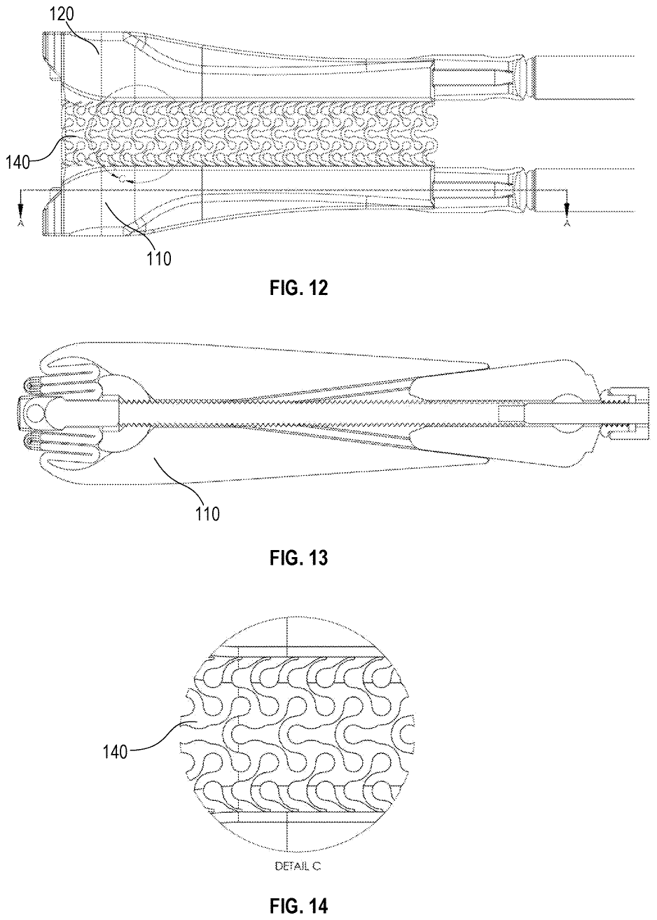

The mesh structure of coupling member 140 may include a series of interconnected curved elements arranged in a wave-like pattern, as depicted in FIGS. 11 and 12. This arrangement may create a network of openings throughout the surface of coupling member 140. These openings may allow for controlled deformation of coupling member 140, enabling adaptation to different loading conditions while maintaining structural integrity. Coupling member(s) 140 may include a plurality of openings suitably sized for the flexibility to distribute force effectively. In some cases, the diameter of the openings may be approximately 0.1 mm to 3 mm. In some cases, the diameter of the opening may be approximately 1 mm. In some cases, the openings may be evenly distributed through coupling member(s) 140.

In some cases, coupling member may include a lattice or porous scaffold, truss, or lateral beams, that may allow bone growth.

In some cases, coupling member 140 may be configured to allow relative movement between first adjustable member 110 and second adjustable member 120. This feature may accommodate different spinous process geometries, enhancing the adaptability of implant 100 to various patient anatomies.

The mesh structure of coupling member 140 may provide spaces for bone growth, as shown in FIG. 14. These spaces may promote fusion between adjacent vertebrae by allowing bone tissue to grow through and around coupling member 140. The size and distribution of these spaces may be optimized to balance mechanical strength with bone growth potential.

In some cases, coupling member 140 may include a mesh layer having an elastic property. This elastic property may allow coupling member 140 to exert a restorative force when compressed. The ability to exert a restorative force may help maintain proper spacing between vertebrae and provide dynamic stabilization to the spine.

Coupling member 140 may be connected to each superior arm of first adjustable member 110 and second adjustable member 120. This configuration may define a cavity between the superior arms that may be configured to receive bone graft material. The ability to incorporate bone graft material may further enhance the fusion-promoting capabilities of implant 100.

In some cases, coupling member 140 may be a unitary structure, formed as a single piece. In other cases, coupling member 140 may include separate components that are connected to form the complete structure. The choice between a unitary or multi-component design may depend on manufacturing considerations, desired mechanical properties, or specific clinical requirements.

Coupling member 140 may be overmolded over a titanium mesh in some cases. This construction technique may combine the strength and biocompatibility of titanium with the formability and customizable properties of an overmolded material. The overmolding process may allow for fine-tuning of the coupling member's mechanical properties and surface characteristics.

Biologic materials may be incorporated into the implant design to promote bone growth and fusion. In some implementations, the spaces within the mesh structure of the coupling member may be filled with bone graft material or other osteogenic substances. This may enhance the implant's ability to achieve long-term stabilization through bony ingrowth.

FIG. 13 illustrates a sectional view (SECTION A-A) of the spinal implant device, revealing a bellows-like structure within the adjustable members. This bellows structure may serve multiple purposes in the implant design, including facilitating the incorporation of biologic materials to promote bone growth and fusion.

The bellows structure may include a series of accordion-like folds or pleats extending along the longitudinal axis of the implant. These folds may be formed within the superior and inferior arms of the adjustable members, creating expandable cavities that can change volume as the implant is adjusted. The bellows design allows for controlled expansion and contraction of the implant while maintaining a sealed internal environment.

In some implementations, the cavities formed by the bellows structure may be used to house biologic materials such as bone graft material, bone morphogenetic proteins (BMPs), or other osteogenic substances. As the implant is expanded, the bellows structure may help to distribute these materials more evenly throughout the implant and into the surrounding tissue.

The expandable nature of the bellows may also allow for the incorporation of a greater volume of biologic material compared to static designs. As the implant is adjusted and the bellows expand, additional space may be created for bone growth, potentially enhancing the implant's ability to achieve long-term stabilization through bony ingrowth.

Furthermore, the bellows structure may work in conjunction with the mesh structure of the coupling member. As the bellows expand, they may exert pressure on the biologic materials, encouraging their distribution through the openings in the mesh. This may help to ensure that the osteogenic substances come into contact with the surrounding bone tissue, promoting fusion between the adjacent vertebrae.

The use of a bellows design may also provide additional benefits such as shock absorption and load distribution, which may contribute to the overall performance and longevity of the implant. The flexible nature of the bellows may allow for some degree of controlled motion, potentially reducing stress on adjacent vertebral segments.

FIG. 3 illustrates components of insertion instrument 600, which may include an elongate rod 602 and an adjustment shaft 604. Elongate rod 602 may include one or more guide rods extending from a posterior end of implant 100. Adjustment shaft 604 may be configured to be inserted into elongate rod 602 to engage and actuate a component of adjustment mechanism 150A.

In some cases, insertion instrument 600 may be configured to deploy implant 100 through a posterior incision of less than 3 centimeters. Insertion instrument 600 may include a handle and an elongated shaft. An actuator on insertion instrument 600 may be rotatably coupled to the handle and operatively connected to adjustment mechanism 150A via the elongated shaft.

Implant 100 may be positioned between adjacent spinous processes to distract and stabilize the vertebrae. In some cases, implant 100 may be inserted in a collapsed or low-profile configuration to facilitate navigation through soft tissues and then transitioned to an expanded configuration in situ. This transition may be achieved by actuating adjustment mechanism 150A to increase or decrease a spacing between the superior arm and the inferior arm at the anterior end of first adjustable member 110 and second adjustable member 120.

The surgical procedure for inserting the spinal implant 100 may involve creating a vertical incision of approximately 1 inch and cutting the interspinous ligament. Implant 100 may then be inserted between the adjacent spinous processes. X-ray imaging may be used to guide the insertion and ensure proper positioning of implant 100.

In some cases, adjustment shaft 604 may be configured to translate first wedge member 155A or second wedge member 156A within implant 100. This translation may increase the spacing between the superior arm and the inferior arm at the anterior end of first adjustable member 110 or second adjustable member 120.

Insertion instrument 600 may include a torque-limiting mechanism configured to prevent over-distraction of implant 100. This mechanism may help ensure that adjustment mechanism 150A is not actuated beyond a safe range, potentially reducing the risk of damage to the patient's anatomy.

During the insertion procedure, at least one inferior arm of first adjustable member 110 or second adjustable member 120 may be positioned in contact with a lamina of a vertebra. This positioning may help distribute loading beyond the spinous processes.

Actuating adjustment mechanism 150A may increase a distance between the superior arms and inferior arms of the first adjustable member 110 and the second adjustable member 120. In some cases, this actuation may expand coupling member 140, which may include a flexible mesh structure. The expansion of coupling member 140 may distribute forces across the adjacent spinous processes.

Adjustment mechanism 150A may be actuated through a posterior surgical approach. This approach may allow for minimally invasive adjustment of implant 100 after insertion. The entire surgical procedure, including insertion and adjustment of implant 100, may be completed in approximately 15 minutes in some cases.

In some embodiments, the implant is configured for placement between adjacent vertebrae, such as within the interspinous space between the spinous processes of adjacent vertebrae. The implant may be positioned using a posterior surgical approach, typically through a small incision (e.g., less than 3 cm), and inserted in a collapsed or low-profile configuration to facilitate navigation through soft tissues.

Following initial positioning between the vertebrae, an adjustment mechanism may be actuated to expand the implant. The adjustment mechanism may include one or more wedge members, such as a first wedge member and a second wedge member, which are configured to translate relative to one another along a longitudinal axis of the implant. In some embodiments, a threaded drive shaft or other surgical instrument may be engaged at a posterior interface and rotated or advanced to translate one wedge member (e.g., the inner wedge) forward with respect to the other.

As the wedge member advances, its inclined surfaces may engage multi-slope inner contours defined by the superior and inferior arms of one or both expansion members. These inner contours may include a series of angled or curved surfaces, such as an entry taper, a primary ramp, a secondary ramp, a plateau or transition region, and a posterior stabilization zone. As the wedge translates, it interacts with these surfaces to incrementally increase the spacing between the superior and inferior arms, thereby producing controlled vertical expansion of the implant.

This expansion results in distraction of the vertebral space—specifically, separating the adjacent spinous processes and/or laminae in a controlled manner. The distraction may decompress neural structures by increasing foraminal height and restoring anatomical spacing lost due to degeneration or collapse.

The wedge-driven expansion mechanism may allow the implant to be fine-tuned in situ, offering the surgeon precise control over the amount and direction of distraction. As distraction occurs, compressive forces from the adjacent vertebrae are received by the outer sidewalls of the superior and inferior arms. These forces may be distributed longitudinally along the arms, laterally across the contact surfaces, and through one or more coupling members, such as a mesh or lattice layer, which spans between the first and second expansion members.

In some embodiments, the coupling member may possess elastic properties that exert a restorative force against compression. This force may maintain distraction even after the wedge member is in its final position and may support gradual adaptation or settling over time.

The implant's design may also resist backward migration of the wedge member after full expansion. For example, a posterior stabilization zone may include a reverse taper or interference feature that provides mechanical resistance to recoil. Additionally, the interaction between nested wedge members may prevent decoupling or shifting under cyclic spinal loads.

Overall, the implant may be configured to convert longitudinal instrument-driven actuation into controlled vertical distraction, while managing compressive and shear forces through carefully shaped surfaces, material interfaces, and structural stabilization features. This may reduce risk of bone damage and promote secure, long-term fixation.

Materials used in the implant devices may include biocompatible metals, polymers, and/or ceramics. In some implementations, porous or mesh structures may be incorporated to further promote bone ingrowth and long-term stabilization. The specific materials and structures may be selected to provide appropriate mechanical properties while supporting biological integration. The adjustable members, coupling members, adjustable members, and insertion instrument may be fabricated from various biocompatible materials, including but not limited to titanium alloys, stainless steel, or polymers such as polyetheretherketone (PEEK). The specific material selection may depend on factors such as desired mechanical properties, biocompatibility, and imaging compatibility.

While the disclosure has been described in connection with what is presently considered to be the most practical and preferred embodiments, it is to be understood that the disclosure is not to be limited to the disclosed embodiments, but on the contrary, is intended to cover various modifications and equivalent arrangements included within the spirit and scope of the appended claims, which scope is to be accorded the broadest interpretation so as to encompass all such modifications and equivalent structures as permitted under the law. Furthermore, it should be understood that while the use of the word preferable, preferably, or preferred in the description above indicates that feature so described may be more desirable, it nonetheless may not be necessary and any embodiment lacking the same may be contemplated as within the scope of the disclosure, that scope being defined by the claims that follow. In reading the claims it is intended that when words such as “a,” “an,” “at least one” and “at least a portion” are used, there is no intention to limit the claim to only one item unless specifically stated to the contrary in the claim. Further, when the language “at least a portion” and/or “a portion” is used the item may include a portion and/or the entire item unless specifically stated to the contrary.

The following clauses illustrate example subject matter described herein.

Clause 1. An implant for placement between adjacent vertebrae spinous processes, comprising: a first adjustable member and a second adjustable member, each comprising: a superior arm and an inferior arm, each having one or more surfaces configured to contact one or more vertebra; an anterior end configured for insertion between adjacent vertebrae; and a posterior end opposite the anterior end, wherein the superior arm and inferior arm define a spacing therebetween, and wherein an anterior spacing at the anterior end is adjustable relative to a posterior spacing at the posterior end; and one or more coupling members extending between and coupled to the first adjustable member and the second adjustable member, the one or more coupling members configured to permit bone growth therethrough; and an adjustment mechanism operably coupled to at least one of the adjustable members and configured to increase or decrease the anterior spacing.

Clause 2. The implant of clause 1, wherein the anterior spacing between the superior arm and the inferior arm is configured to have a maximum spacing at the anterior end and the posterior spacing between the superior arm and the inferior arm is configured to have a minimum spacing at the posterior end.

Clause 3. The implant of clauses 1 or 2, wherein the adjustment mechanism comprises a threaded screw, ratcheting element, or cam configured to alter the anterior spacing between the superior arm and the inferior arm.

Clause 4. The implant of any one of clauses 1 through 3, wherein the one or more coupling members comprise at least one of a mesh, a lattice, or a porous scaffold.

Clause 5. The implant of any one of clauses 1 through 4, wherein a material of the one or more coupling members comprises at least one of titanium, metals suitable for osseointegration, PEEK, polymers suitable for osseointegration, and a resorbable polymer.

Clause 6. The implant of any one of clauses 1 through 5, wherein a first coupling member of the one or more coupling members is connected to each of the superior arms of the first and second adjustable members and define between the superior arms a cavity configured to receive bone graft material.

Clause 7. The implant of any one of clauses 1 through 6, wherein the superior arm and the inferior arm of at least one of the adjustable members define a wedge-shaped profile with the anterior spacing at the anterior end being greater than the posterior spacing at the posterior end, wherein the superior arm and the inferior arm each define an inclined surface positioned on opposite sides of a central axis extending between the superior arm and the inferior arm, and wherein the adjustment mechanism comprises a first wedge member and a second wedge member, the first wedge member and the second wedge member being configured to translate relative to one another along the inclined surfaces to increase the anterior spacing between the superior arm and the inferior arm at the anterior end.

Clause 8. The implant of any one of clauses 1 through 7, wherein the superior arm and the inferior arm of at least one of the adjustable members each comprise a multi-slope inner contour comprising at least an entry taper having a first inclined surface and a primary ramp having a second inclined surface, and the first inclined surface is less steep than the second inclined surface relative to a longitudinal axis of the implant, the first and second inclined surfaces being configured to sequentially engage a wedge member during actuation of the adjustment mechanism.

Clause 9. The implant of any one of clauses 1 through 8, wherein first and second adjustable members are configured to receive and distribute forces from adjacent spinous processes, and the one or more coupling members are configured to distribute forces from the first adjustable member and the second adjustable member.

Clause 10. The implant of any one of clauses 1 through 9, wherein at least one of the inferior adjustable member is configured to contact a lamina of a vertebra to distribute loading beyond the spinous processes.

Clause 11. A method for implanting a spinal implant between adjacent vertebrae spinous processes, the method comprising: inserting an implant between the adjacent spinous processes, the implant comprising: a first adjustable member and a second adjustable member, each comprising a superior arm and an inferior arm, an anterior end, and a posterior end; one or more coupling members extending between and coupled to the first adjustable member and the second adjustable member; and an adjustment mechanism operably coupled to at least one of the adjustable members; and actuating the adjustment mechanism to increase or decrease an anterior spacing between the superior arm and the inferior arm at the anterior end.

Clause 12. The method of clause 11, further comprising placing bone graft material within the one or more coupling members before or after implantation.

Clause 13. The method of clauses 11 or 12, wherein the adjustment mechanism is actuated through a posterior surgical approach.

Clause 14. The method of any one of clauses 11 through 13, wherein the implant is transitioned from a collapsed configuration to an expanded configuration in situ.

Clause 15. The method of any one of clauses 11 through 14, wherein the implant is positioned between adjacent spinous processes to distract and stabilize the vertebrae.

Clause 16. A system for stabilizing adjacent vertebrae, comprising: an implant configured for placement between adjacent vertebrae, the implant comprising: a first adjustable member and a second adjustable member, each comprising a superior arm and an inferior arm, an anterior end, and a posterior end; one or more coupling members extending between and coupled to the first adjustable member and the second adjustable member; and an adjustment mechanism operably coupled to at least one of the adjustable members and configured to adjust an anterior spacing at the anterior end; and an instrument configured to engage the implant and actuate the adjustment mechanism during implantation.

Clause 17. The system of clause 16, wherein the instrument comprises a torque-limiting mechanism configured to prevent over-distraction of the implant.

Clause 18. The system of clauses 16 or 17, wherein the instrument comprises one or more elongate guide rods extending from a posterior end of the implant and an elongate actuator configured to be inserted into the guide rods to engage and actuate a component of the adjustment mechanism.

Clause 19. The system of any one of clauses 16 through 18, wherein the instrument is configured to deploy the implant through a posterior incision of less than 3 centimeters.

Clause 20. The system of clause 18, wherein the elongate actuator is configured to translate a wedge member within the implant to increase the anterior spacing between the superior arm and the inferior arm at the anterior end.

Clause 21. The system of any one of clauses 16 through 20, wherein the implant further comprises textured outer surfaces to enhance bony fixation.

Clause 22. The implant of any one of clauses 1 through 10, wherein the one or more coupling members comprise a mesh layer having an elastic property configured to exert a restorative force when compressed.

Clause 23. The implant of clause 22, wherein the mesh layer is configured to continually distract the disc space following implantation.

Clause 24. The implant of clauses 22 or 23, wherein the mesh layer is configured to undergo gradual compression or relaxation over a period of minutes to hours following implantation.

Clause 25. A spinal implant device, comprising: a first adjustable member and a second adjustable member, each having a superior arm and an inferior arm; an adjustment mechanism coupled to the first and second adjustable members and configured to adjust a distance between the superior and inferior arms of each adjustable member; and a flexible mesh structure spanning between the first and second adjustable members, wherein the flexible mesh structure is configured to distribute forces across spinous processes of adjacent vertebrae when implanted.

Clause 26. The spinal implant device of clause 25, wherein the adjustment mechanism comprises a threaded shaft and a nut, the threaded shaft configured to engage with the nut to adjust the distance between the superior and inferior arms.

Clause 27. The spinal implant device of clauses 25 or 26, wherein the adjustment mechanism further comprises a first wedge member and a second wedge member, the first and second wedge members configured to move along the threaded shaft to adjust the distance between the superior and inferior arms.

Clause 28. The spinal implant device of any one of clauses 25 through 27, wherein the flexible mesh structure comprises a titanium mesh.

Clause 29. The spinal implant device of any one of clauses 25 through 28, wherein the superior and inferior arms of each adjustable member comprise textured surfaces configured to engage with the spinous processes.

Clause 30. The spinal implant device of any one of clauses 25 through 29, further comprising a coupling member connecting the first adjustable member to the second adjustable member.

Clause 31. The spinal implant device of clause 30, wherein the coupling member is configured to allow relative movement between the first and second adjustable members to accommodate different spinous process geometries.

Clause 32. A method of implanting a spinal device between adjacent spinous processes, comprising: inserting the spinal device between the adjacent spinous processes, the spinal device including first and second adjustable members connected by a flexible mesh structure; and actuating an adjustment mechanism to increase a distance between superior and inferior arms of each adjustable member, thereby expanding the flexible mesh structure to distribute forces across the adjacent spinous processes.

Clause 33. The method of clause 32, wherein actuating the adjustment mechanism comprises rotating a threaded shaft to move wedge members that increase the distance between the superior and inferior arms.

Clause 34. The method of clause 33, wherein rotating the threaded shaft causes the wedge members to move in opposite directions along the longitudinal axis of the spinal device.

Clause 35. The method of any one of clauses 32 through 34, further comprising packing a biologic material within the flexible mesh structure to promote bone growth between the adjacent spinous processes.

Clause 36. The method of clause 35, wherein the biologic material comprises bone graft material.

Clause 37. The method of any one of clauses 32 through 36, further comprising adjusting the relative positions of the first and second adjustable members to accommodate different spinous process geometries.

Clause 38. The method of clause 37, wherein adjusting the relative positions comprises sliding a coupling member connecting the first and second adjustable members along a longitudinal axis of the spinal device.

Clause 39. A system for spinal stabilization, comprising: an implantable device having first and second adjustable members connected by a flexible mesh structure; an insertion instrument removably coupled to the implantable device; and an actuator on the insertion instrument configured to engage an adjustment mechanism of the implantable device to adjust an anterior spacing between superior and inferior arms of the first and second adjustable members.

Clause 40. The system of clause 39, wherein the flexible mesh structure comprises a titanium mesh configured to distribute forces across spinous processes of adjacent vertebrae when implanted.

Clause 41. The system of clauses 39 or 40, wherein the adjustment mechanism comprises a threaded shaft and a nut, the threaded shaft configured to engage with the nut to adjust the anterior spacing between the superior and inferior arms.

Clause 42. The system of any one of clauses 39 through 41, wherein the adjustment mechanism further comprises a first wedge member and a second wedge member, the first and second wedge members configured to move along the threaded shaft to adjust the anterior spacing between the superior and inferior arms.

Clause 43. The system of clause 42, wherein rotation of the actuator causes the wedge members to move in opposite directions along a longitudinal axis of the implantable device.

Clause 44. The system of any one of clauses 39 through 43, wherein the insertion instrument comprises a handle and an elongated shaft, and wherein the actuator is rotatably coupled to the handle and operatively connected to the adjustment mechanism via the elongated shaft.

What is claimed is:

1. An implant for placement between adjacent vertebrae, comprising:
a first adjustable member and a second adjustable member, each of the first adjustable member and the second adjustable member comprising:
a superior arm and an inferior arm, each of the superior arm and the inferior arm having one or more surfaces configured to contact one or more vertebrae;
an anterior end configured for insertion between the adjacent vertebrae; and
a posterior end opposite the anterior end,
wherein the superior arm and the inferior arm define an anterior spacing at the anterior end and a posterior spacing at the posterior end, and
wherein the anterior spacing is adjustable relative to the posterior spacing;
one or more coupling members extending between and coupled to the first adjustable member and the second adjustable member, the one or more coupling members configured to permit bone growth therethrough; and
an adjustment mechanism operably coupled to at least one of the first adjustable member and the second adjustable member and configured to increase or decrease the anterior spacing.

2. The implant of claim 1, wherein the anterior spacing is a maximum spacing and the posterior spacing is a minimum spacing.

3. The implant of claim 1, wherein the adjustment mechanism comprises at least one of a threaded screw, a ratcheting element, or a cam configured to alter the anterior spacing.

4. The implant of claim 1, wherein the one or more coupling members comprise at least one of a mesh, a lattice, or a porous scaffold.

5. The implant of claim 1, wherein the one or more coupling members comprise at least one of titanium, PEEK, or a resorbable polymer.

6. The implant of claim 1, wherein a first coupling member of the one or more coupling members is connected to the superior arm of the first adjustable member and the superior arm of the second adjustable member and defines a cavity between the superior arm of the first adjustable member and the superior arm of the second adjustable member, the cavity configured to receive bone graft material.

7. The implant of claim 1, wherein the superior arm and the inferior arm of at least one of the first adjustable member and the second adjustable member define a wedge-shaped profile with the anterior spacing being greater than the posterior spacing, and wherein the adjustment mechanism comprises a first wedge member and a second wedge member configured to translate relative to one another along opposing inclined surfaces to increase the anterior spacing.

8. The implant of claim 1, wherein the superior arm and the inferior arm of at least one of the first adjustable member and the second adjustable member each comprise a multi-slope inner contour comprising at least an entry taper having a first inclined surface and a primary ramp having a second inclined surface, the first inclined surface being less steep than the second inclined surface relative to a longitudinal axis of the implant, the first inclined surface and the second inclined surface being configured to sequentially engage a wedge member of the adjustment mechanism.

9. The implant of claim 1, wherein the first adjustable member and the second adjustable member are configured to receive and distribute forces from adjacent spinous processes, and the one or more coupling members are configured to distribute forces between the first adjustable member and the second adjustable member.

10. The implant of claim 1, wherein at least one of the inferior arm of the first adjustable member or the inferior arm of the second adjustable member is configured to contact a lamina of a vertebra to distribute loading beyond spinous processes of the adjacent vertebrae.

11. A method for implanting a spinal implant between adjacent vertebrae, the method comprising:
inserting an implant between adjacent spinous processes of the adjacent vertebrae, the implant comprising:
a first adjustable member and a second adjustable member, each of the first adjustable member and the second adjustable member comprising a superior arm and an inferior arm, an anterior end, and a posterior end;
one or more coupling members extending between and coupled to the first adjustable member and the second adjustable member; and
an adjustment mechanism operably coupled to at least one of the first adjustable member and the second adjustable member;
actuating the adjustment mechanism to increase or decrease an anterior spacing between the superior arm and the inferior arm at the anterior end; and
positioning at least one of the inferior arm of the first adjustable member or the inferior arm of the second adjustable member in contact with a lamina of a vertebra.

12. The method of claim 11, further comprising placing bone graft material within the one or more coupling members.

13. The method of claim 11, wherein actuating the adjustment mechanism comprises actuating the adjustment mechanism through a posterior surgical approach.

14. The method of claim 11, further comprising transitioning the implant from a collapsed configuration to an expanded configuration in situ by actuating the adjustment mechanism.

15. The method of claim 11, wherein actuating the adjustment mechanism comprises rotating a threaded shaft to translate a first wedge member and a second wedge member in opposite directions along a longitudinal axis of the implant to increase the anterior spacing.

16. A system for stabilizing adjacent vertebrae, comprising:
an implant configured for placement between adjacent spinous processes of the adjacent vertebrae, the implant comprising:
a first adjustable member and a second adjustable member, each of the first adjustable member and the second adjustable member comprising a superior arm and an inferior arm, an anterior end, and a posterior end;
one or more coupling members extending between and coupled to the first adjustable member and the second adjustable member, the one or more coupling members configured to permit bone growth therethrough; and
an adjustment mechanism operably coupled to at least one of the first adjustable member and the second adjustable member and configured to adjust an anterior spacing at the anterior end; and
an instrument configured to engage the implant and actuate the adjustment mechanism during implantation.

17. The system of claim 16, wherein the instrument comprises a torque-limiting mechanism configured to prevent over-distraction of the implant.

18. The system of claim 16, wherein the instrument comprises one or more elongate guide rods extending from the posterior end of the implant and an elongate actuator configured to be inserted into the one or more elongate guide rods to engage and actuate the adjustment mechanism.

19. The system of claim 18, wherein the elongate actuator is configured to translate a wedge member within the implant to increase the anterior spacing.

20. The system of claim 16, wherein the one or more coupling members comprise a mesh layer having an elastic property configured to exert a restorative force when compressed.
